(19) United States
(12) Patent Application Publication
(10) Pub. No.: US 2011/0118344 A1
(43) Pub. Date: May 19, 2011

(54) Novel compound having inhibitory effect on lipase

(76) Inventors: Masaaki Nakai, Osaka (JP); Yuko Fukui, Osaka (JP); Sumio Asami, Osaka (JP)

(21) Appl. No.: 11/630,132

(22) PCT Filed: Jun. 20, 2005
(86) PCT No.: PCT/JP05/11258

§ 371 Date: Nov. 30, 2007

(30) Foreign Application Priority Data
Jun. 21, 2004 (JP) 2004-182471

(51) Int. Cl. A61K 31/352; C07D 407/14; C12P 17/16; A61P 3/06; A61P 3/04

(52) U.S. Cl. 514/456; 549/399; 435/118

(57) ABSTRACT

This invention provides, a novel polyphenol compound having lipase inhibitory activity derived from tealeaves, a process for the preparation of the compound, and foods and/or beverages and pharmaceutical drugs containing the compound.

Particularly, this invention provides a novel dimer compound of formula:

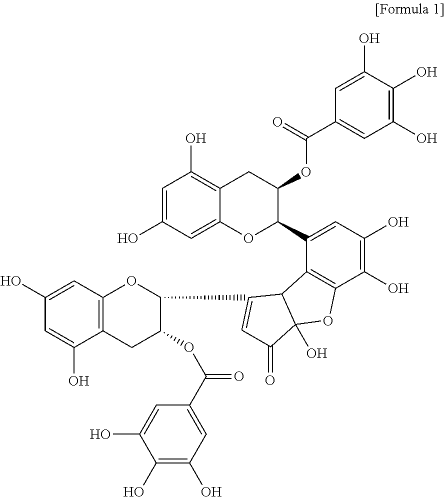

obtained by oxidative polymerization of epigallocatechin-3-O-gallate, a main catechin component of oolong tea, with a tealeaf enzyme (polyphenol oxidase), as well as foods and/or beverages and pharmaceutical compositions containing said novel compound which suppress absorption of dietary lipids and suppresses increase of blood triglyceride.

### TECHNICAL FIELD

This invention provides, a novel polyphenol extracted from oolong tea having lipase inhibitory activity, a process for its preparation, and foods and/or beverages and pharmaceutical compositions containing the polyphenol.

### BACKGROUND ART

With the recent tendency toward westernized eating habits in Japan, intake of high fat diet continues to increase. According to a National Nutrition Survey in Japan (1999) among people over 60, it is reported that although their energy intake is decreasing every year, their fat energy ratio exceeds the reasonable proportion of 25%, and 50 to 60% of those people are recognized to have high triglyceride and cholesterol values [A Summary of 1999 National Nutrition Survey in Japan by The Ministry of Health, Labor and Welfare, Rinsho Eiyo (Clinical nutrition) 2001; 98(5): 577-588].

Obesity is one of the most severe diseases in present day society, caused by excessive fat intake. The excessive fat intake causes not only obesity, but also contracting disorders such as diabetes, hyperlipidemia, hypertension and arteriosclerosis. In Japan, Mazindole (registered trademark) as an anorectic drug is only one therapeutic drug with official approval for treating obesity. However, this drug is reported to have side effects such as excessive thirst (mouth dryness), constipation, epigastric distress, nausea and vomiting [Rinsyo Hyouka (Clinical evaluation), 1985; 13(2): 419-459, Clinical evaluation, 1985; 13(2): 461-515]. In overseas, Xenical (registered trademark) as a lipase inhibitor which suppresses fat absorption in the gastrointestinal tract, is on market as an obesity treatment drug. However, this drug is also reported to have side effects such as fatty stool, increased stool frequency, soft stool, diarrhea and stomachache. Therefore, using this drug is sometimes accompanied by concerns about safety (The Lancet 1998; 352:67-172) .

To prevent obesity, it is advantageous to reduce the caloric intake by controlling diet. However, it requires careful guidance on nutrition making it difficult to practice in daily life. Therefore, inhibiting the absorption of dietary lipids in the body in a safe and healthy manner is practical and useful for treatment of obesity and related diseases and in promoting health.

With these facts in mind, the development of a “food for specified health uses” which is safe to use and is proven to be effective in treating humans is attracting a lot of attention. Food materials which inhibit increase of serum triglyceride after a meal, such as: a globin protein decomposition product that suppresses fat absorption by pancreatic lipase inhibitory activity [J. Nutr. 1988; 128: 56-60, 1988, Nihon Eiyou Shokuryou Gakkai-shi (Journal of Japanese society of Nutrition and Food Science) 1999; 52(2): 71-77, Kenkou Eiyou Shokuhin Kenkyu (Health food and nutrition food Research) 2002; 5(3): 131-144]; diacylglycerol with different digestion and absorption features compared to triacylglycerol (J. Am. Coll. Nutr. 2000; 19(6): 789-796, Clin. Chim. Acta. 2001; 11(2): 109-117); eicosapentaenoic acid (EPA) and docosahexanoic acid (DHA) purified from fish oil; are on market as foods for specified health use until now.

On the other hand, lipase inhibitors derived from plants are also attracting attention in recent years. Especially, as to the polyphenols with lipase inhibitory activity, for example: tannin from bark (JP Shou 60-11912-B); tannins, flavonoids and glucosides thereof from leguminous plant (Cassia mimosoides L.var.nomame Makino) (JP Hei 8-259557-A); epigallocatechin gallate which is the main component in green tea, and lipid absorption suppressing food containing the epigallocatechin gallate (JP Hei 3-228664-A); lipase inhibitory agent containing water extracts from green pepper, shimeji mushrooms, pumpkin, Grifola frondosa (maitake), Hizikia fusiforme, green tea, oolong tea, and others (JP Hei 3-219872-A); flavons and flavonols (JP Hei 7-61927-A); hydroxybenzoic acids (gallic acid) (JP Hei 1-102022-A); triterpenes and derivatives thereof (JP Hei 9-40689-A) ; and anti-obesity medicine containing procyanidin from Tamarind as an active ingredient (JP Hei 9-291039-A) are reported. Also, the lipase inhibitory effect of grape seed extract (Nutrition vol.19, (10), 876-879,2003), the lipase inhibitory effect and anti-obesity effect of polyphenol from Salacia in rats (J. Nutr., 132, 1819-1824,2002), anti-obesity effect of oolong tea extract in rats (Int. J. Obes., 23 98-105, 1999), and others are known.

However, reported lipase inhibitory agents from plants mentioned above are not sufficiently effective. For instance, since they originate from natural sources, there is a problem in maintaining stable lipase inhibitory activity when the content of the active ingredient in the plant is not clearly known. Moreover, an inhibitory agent derived from plants with less preference will raise a flavor problem when used in foods and/or beverages. For example, reports on lipid-improving effect of oolong tea are: significant decrease in blood triglyceride after drinking 1330 ml/day of commercial oolong tea for 6 weeks [Nihon Eiyou Shokuryou Gakkai-shi (Journal of Japanese society of Nutrition and food science) 1991; 44(4): 251-259]; and oral administration of oolong tea (2 g×4/day) for 6 consecutive weeks to 102 males and females with simple obesity resulted in more than 1 kg weight loss in 67% of the subjects and significant improvement in the subjects with high blood acylglycerol after taking oolong tea [Nihon Rinsho Eiyou Gakkai-shi (The Japanese Society of Clinical Nutrition Magazine) 1998; 20(1): 83-90]. These reports show that although drinking a large quantity of oolong tea is recognized to be effective, it is difficult in daily life to continue drinking such large quantities of a drink such as oolong tea. Further, simply providing concentrated oolong tea is not an appropriate and a practical option, due to its strong bitterness and astringency and increased caffeine content.

### Patent Documents

1. JP Shou 60-11912-B
2.JP Hei 8-259557-A
3.JP Hei 3-228664-A
4. JP Hei 3-219872-A
5.JP Hei 7-61927-A
6. JP Hei 1-102022-A
7. JP Hei 9-40689-A
8.JP Hei 9-291039-A

### Non Patent Documents

1. A Summary of 1999 National Nutrition Survey in Japan by The Ministry of Health, Labor and Welfare
2.Rinsho Eiyo (Clinical nutrition) 2001; 98(5): 577-588
3.Rinsyo Hyouka (Clinical evaluation), 1985; 13(2): 419-459, Clinical evaluation, 1985; 13(2): 461-515
4. The Lancet 1998; 352:67-172
5. J. Nutr. 1988; 128: 56-60, 1988
6. Nihon Eiyou Shokuryou Gakkai-shi (Journal of Japanese society of Nutrition and Food Science) 1999; 52(2): 71-77
7. Kenkou Eiyou Shokuhin Kenkyu (Health food and nutrition food Research) 2002; 5(3): 131-144
8. J. Am. Coll. Nutr. 2000; 19(6): 789-796
9. Clin. Chim. Acta. 2001; 11(2): 109-117
10. Nutrition vol.19, (10), 876-879,2003
11.J. Nutr., 132, 1819-1824, 2002
12.Int. J. Obes., 23 98-105, 1999
13. Nihon Eiyou Shokuryou Gakkai-shi (Journal of Japanese society of Nutrition and food science) 1991; 44(4): 251-259
14. Nihon Rinsho Eiyou Gakkai-shi (The Japanese Society of Clinical Nutrition Magazine) 1998; 20(1): 83-90

### DISCLOSURE OF THE INVENTION

### Problem to be Solved by the Invention

The present invention targets particular components in tea that has high preference, and provides a novel polyphenol present in oolong tea having lipase inhibitory activity, and a process for its preparation.

The present invention further provides foods and/or beverages containing the novel polyphenol compound of the invention having lipase inhibitory activity, which will suppress the absorption of dietary lipids and suppress the rise of triglyceride in blood.

The present invention further provides a pharmaceutical composition containing the novel polyphenol compound of the invention having lipase inhibitory activity which will suppress the absorption of dietary lipids and suppress the rise of triglyceride in blood.

### Means for Solving Problem

The inventors found that oxidative polymerization of epigallocatechin-3-O-gallate with a tea leaf enzyme (polyphenol oxidase) gives a novel dimer compound, oolongtheanin-3′-O-gallate of the following formula:

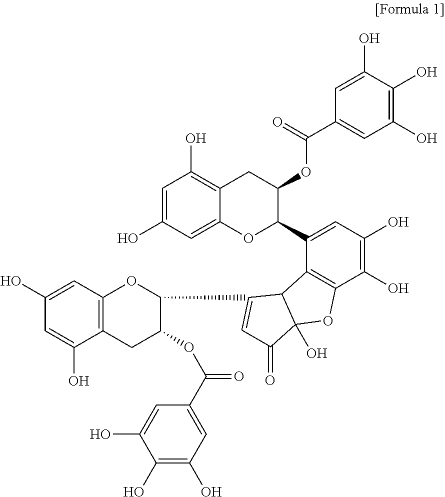

which has a strong inhibitory effect on pancreatic lipase, an enzyme essential for fat absorption.

Process for preparation

The compound of the present invention can be obtained by oxidative polymerization of epigallocatechin-3-O-gallate with polyphenol oxidase. Starting material epigallocatechin-3-O-gallate is a known compound which is commercially available. Also, it can be obtained from natural materials such as green tea, black tea, and oolong tea by extraction. Polyphenol oxidase used for oxidative polymerization can be prepared by, for example, extraction from tealeaf according to the process described in Example 1. However, it is not limited to enzymes of tealeaf origin as long as it catalyzes the oxidative polymerization reaction of epigallocatechin-3-O-gallate into oolongtheanin-3′-O-gallate (OTNG). For example, an enzyme derived from horseradish can be used.

The oxidative polymerization reaction is carried out by placing a starting material (epigallocatechin-3-O-gallate), an oxidiant (for example, H₂O₂), and polyphenol oxidase in an aqueous buffer solution with pH 4 to 7, preferably pH 5 to 6, at 20 to 40° C., preferably 25 to 35° C., for 1 to 4 hours, preferably 3 hours. For 100 mg of starting material, for example, 2 mg of oxidant and polyphenol oxidase obtained from 100 g of fresh tealeaf, can be used respectively.

The product obtained from oxidative polymerization reaction may be purified by conventional methods such as chromatography. Purified oolongtheanin-3′-O-gallate is a “white powder, soluble in water, methanol and DMSO, and neutral”, which is very safe. Therefore, this compound is suitable for use in, for example, foods and/or beverages, and pharmaceutical drugs as an active lipase inhibitory ingredient for suppressing absorption of dietary lipids, thereby suppressing the rise of blood triglyceride or decreasing increased blood triglyceride.

Although oolongtheanin-3′-O-gallate is provided as a novel compound as a result of the above oxidative polymerization reaction, there is a possibility that it is present in natural materials such as tealeaves. Therefore, the compound may be obtained from those natural materials by extraction and purification.

### Method for Lipase Inhibitory Activity Determination

The compound of the present invention has strong inhibitory activity against lipase, particularly pancreatic lipase. This inhibitory activity can be determined by the method specifically described in Example 2.

### Lipase Inhibitor

The compound of the present invention may be used as a lipase inhibitor either alone or with a solvent or a solid carrier. Preferably, the solvent or carrier is safe to use in foods or medicament, considering its use in foods and/or beverages and/or medicament mentioned below. The lipase inhibitor of the present invention can be used for various purposes, including, for example, experimental purposes, and as an active ingredient of preventing accumulation of triglyceride in foods and medicaments.

Foods and/or Beverages Containing Oolongtheanin-3′-O-Gallate

The compound or the lipase inhibitory agent containing the compound of the invention may be added to foods and/or beverages as an active lipase inhibitory ingredient in order to prevent undesirable rise of blood triglyceride that may accompany fat intake from diet, and/or to decrease increased blood triglyceride levels. Preferable examples of foods and/or beverages are daily taken foods and/or beverages, such as green tea, mugi-cha (barley tea), oolong tea, black tea, coffee, sports drink, drinking water, seasoning and dressing. However, the foods and/or beverages may be any of those taken usually, such as a soft drink, cocktail, beer, whisky, shochu (rough distilled spirits), wine, sake, seasoning, dressing, flavored rice, processed food, instant food, retort pouch (specially packaged food that has been pre-heated and sterilized), chocolate, fresh cream, confectionery, dairy products (nyu-seihin), health foods, and dietary supplements.

The compound of the present invention is added to foods and/or beverages to provide 0.1 mg to 1000 mg of intake per meal. Provided that since the compound of the present invention derives from food, it is very safe, and there is no practical upper limit of the amount which can be added to foods and/or beverages.

### A Pharmaceutical Drug Containing Oolongtheanin-3′-O-Gallate

The compound or the lipase inhibitory agent of the present invention can also be used as an active ingredient in a drug for suppressing absorption of dietary lipids and preventing and/or decreasing undesirable increase of blood triglyceride. Preferable drugs are drugs suitable for oral administration, such as drinks, tablets, capsules, granules, powders, candies and drops. The drugs comprise the compound of the present invention in amounts of 0.1 mg to 1000 mg per dose.

Since the active lipase inhibitory ingredient oolongtheanin-3′-O-gallate is very safe, the pharmaceutical drug of the present invention can be administered for a long time without the risk of side effects. Therefore, it may be taken daily for the purpose of preventing or treating obesity as a life-style disease.

### Effect of the Invention

By adding polyphenol derived from oolong tea, the present invention provides foods and/or beverages of high preference for reducing triglyceride and promoting health without spoiling flavor. In order to inhibit absorption of dietary lipids, it is desirable to take it with meal. Therefore, beverages enriched with the active ingredient obtained from tea are highly significant.

The compound of the present invention is prepared by a simple process with epigallocatechin-3-O-gallate as a starting material, which is present in large amounts in oolong tea. The purification process is also easy.

### BRIEF DESCRIPTION OF THE DRAWINGS

FIG. 1 shows MS spectrum of Oolongtheanin-3′-O-gallate (OTNG).

FIG. 2 shows ¹H NMR data of OTNG.

FIG. 3 shows ¹³C NMR data of OTNG.

FIG. 4 shows chemical structure of OTNG.

### EXAMPLES

### Example 1

### Enzymatic Synthesis of Oolongtheanin-3′-O-gallate (OTNG)

### Enzyme Preparation

600 g of Tealeaves, Kyoken No. 129 (provided from Kyoto Prefectural Tea Industry Research Institute) was triturated in liquid nitrogen. 1800 ml of extraction buffer (adjusted to pH7.0 with 0.01M KH₂PO₄ and 0.02M K₂HPO₄) and 300 g of polyamide were added and stirred, then filtered through gauze. The filtrate was centrifuged for 20 minutes at 8000 rpm. 1500 ml of acetone cooled to −20° C. in advance was added to 1500 ml of the supernatant, and the mixture was left to stand at 4° C. for 1 hour. The solution was centrifuged at 8000 rpm for 20 minutes at 4° C., to obtain a white precipitate. The precipitate was dissolved in 600 ml of a reaction buffer (adjusted to pH 5.6 with 0.01M citric acid and 0.02M KH₂PO₄) to obtain an enzyme solution.

Enzyme Reaction 600 mg of epigallocatechin-3-O-gallate (Wako Pure Chemical Industries, Ltd.) and 8.8 mM of H₂O₂ were added to 600 ml of the enzyme solution. After stirring, the reaction took place at 32° C. After 3 hours, 600 ml of 90% acetonitrile containing 1% trifluoroacetic acid (TFA) was added to terminate the reaction. The solution was diluted 5-fold with water and applied to absorption resin HP-20 (1000 ml, Mitsubishi Chemical Corporation). After washing with water, the reaction product was eluted with 2000 ml of 90% acetonitrile containing 0.1% TFA. The reaction product was concentrated under reduced pressure, and then lyophilized. The lyophilized product was purified by the following preparative HPLC.

### Purification

Column: Develosil ODS-UG-5 (50 mmφ×500 mm, Nomura Chemical)
Mobile phase: A: 0.05% TFA/H₂O, B:90% CH₃CN, 0.05% TFA

### Detection: A280 nm

Flow rate: 32 ml/min
Gradient: linear gradient elution from B20% to B50% for 100 min.

Oolongtheanin-3′-O-gallate was derived by chromatography and at an elution time of 52 minutes. Another preparative HPLC was carried out for further purification.

Column: Develosil C30-UG-5 (20 mmφ×250 mm, Nomura Chemical)
Mobile phase: A: 0.1% TFA/H₂O, B:90% CH₃CN, 0.1% TFA

### Detection: A280 nm

Flow rate: 6 ml/min
Gradient: linear gradient elution from B10% to B40% for 40 min.

This chromatography gave 25mg of oolongtheanin-3′-O-gallate at an elution time of 34 minutes.

The isolation of oolongtheanin (no gallate) from tealeaves has been reported in Chem.Pharm.Bull 36(5), 1676-1684, 1988. However, the oolongtheanin-3′-O-gallate obtained in Example 1 is a novel compound.

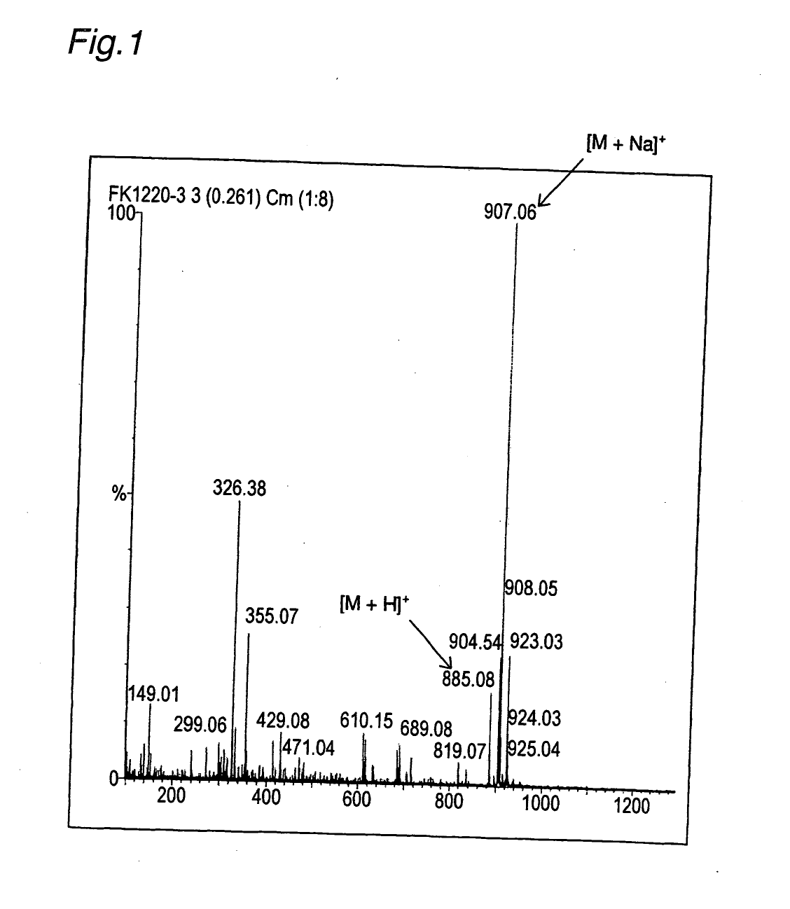

MS was measured with Q-TOF (Micromass, Manchester, UK) using ESI probe, in a positive mode. Ion peaks were observed at m/z 885 for [M+H]⁺ and at m/z 907 for [M+Na]⁺. The spectrum is shown in FIG. 1.

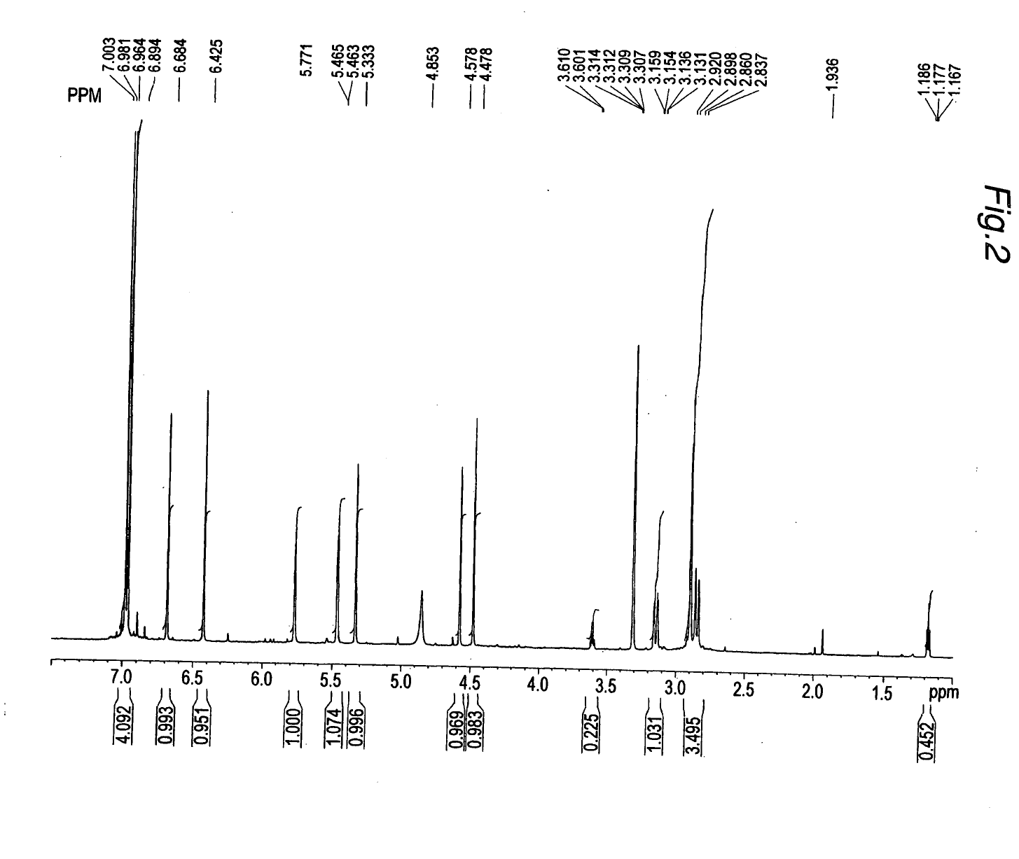

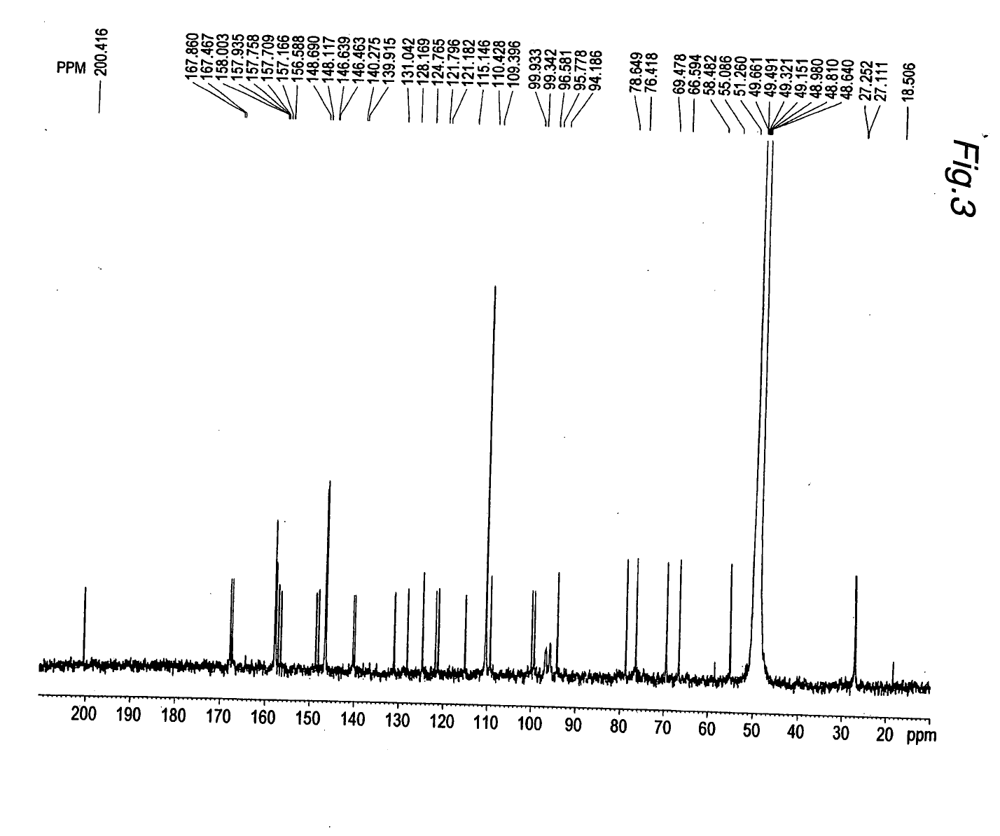

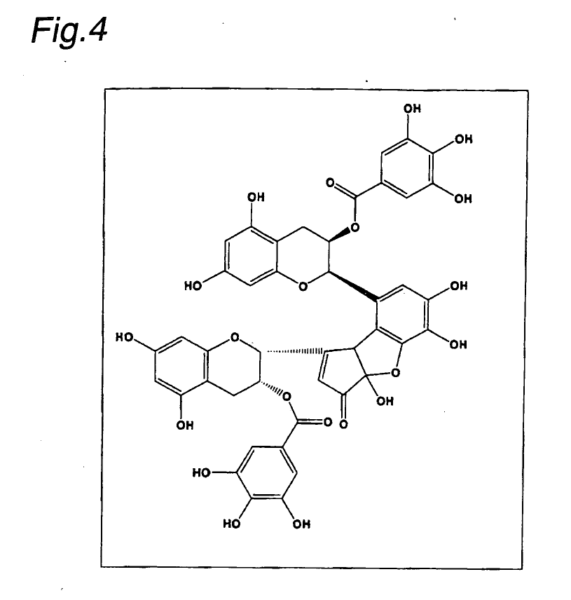

¹H NMR, ¹³C NMR, ¹H{¹³C}-HSQC, ¹H{¹³C}-HMBC, TOCSY, and DQF-COSY were measured with DMX-750 (BRUKER BIOSPIN) in CD₃OD. ¹H NMR and ¹³C NMR are shown in FIGS. 2 and 3, respectively. The structural formula is shown in FIG. 4.

### Example 2

### Lipase Inhibitory Activity Measurement

Lipase activity measurement was carried out by using oleic acid ester of fluorescent 4-methylumbelliferone (4-UMO) as a substrate, and measuring the fluorescence of 4-methylumbelliferone produced by reaction.

In the measurement, 13 mM Tris-HCl containing 150 mM NaCl and 1.36 mM CaCl₂ was used as a buffer (pH 8.0). Substrate 4-UMO (Sigma) was prepared as 0.1M solution in DMSO and diluted 1000-fold with the buffer mentioned above. Similarly, lipase (porcine pancreatic lipase (Sigma)) was prepared as 400 U/ml solution in the buffer mentioned above and used in enzymatic measurement.

50 μl of the 4-UMO buffer solution and 25 μl of distilled water (or sample solution) were placed in a 96-well microplate and mixed at 25° C., followed by adding 25 gl of the lipase buffer solution to start enzyme reaction. After 30 minutes of reaction, 100 μl of 0.1M citric acid buffer (pH 4.2) was added to terminate the reaction, and the fluorescence of 4-methylumbelliferone (excitation wavelength: 355 nm, fluorescence wavelength: 460 nm) produced by the reaction was measured with a fluorescence plate reader (Labsystems, Fluoroskan Asent CF).

An inhibitory activity of the sample was determined as IC₅₀, or the amount of the sample which gave 50% of inhibition compared to the activity of control (distilled water). As to the lipase inhibitory activity of OTNG, its IC₅₀ was 0.06 μg/ml (0.068 μM), showing it to be extremely high in activity compared to EGCG monomer whose IC₅₀ was 0.16 μg/ml (0.349 MM).

1. Oolongtheanin-3′-O-gallate of formula:

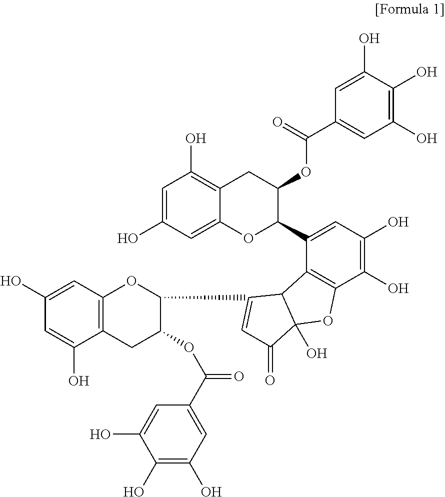

2. A Lipase inhibitory agent containing the oolongtheanin-3′-O-gallate of claim 1.

3. A food or beverage with the oolongtheanin-3′-O-gallate of claim 1 or the lipase inhibitory agent of claim 2.

4. The food or beverage of claim 3 which is selected from the group consisting of tea beverages, soft drinks and health foods.

5. A pharmaceutical composition containing the oolongtheanin-3′-O-gallate of claim 1 or the lipase inhibitory agent of claim 2.

6. The pharmaceutical composition of claim 5 for suppressing absorption of dietary lipids.

7. A process for preparation of oolongtheanin-3′-O-gallate of formula: 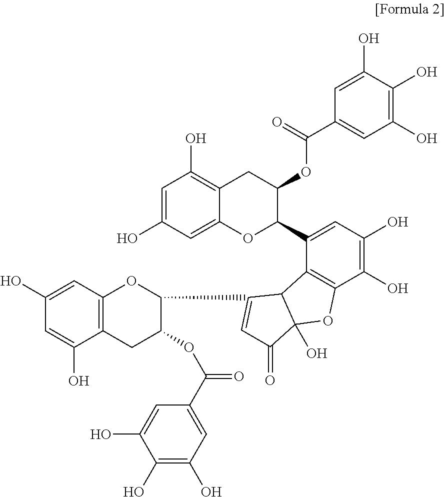
by oxidative polymerization of epigallocatechin-3-O-gallate with polyphenol oxidase in the presence of an oxidant.

8. The process of claim 7 wherein the polyphenol oxidase is derived from tea leaves.

9. The process of claim 7, wherein the oxidative polymerization reaction is carried out at pH 4 to 7, at 20 to 40° C., and with hydrogen peroxide as the oxidant.

10. The food or beverage of claim 3, wherein the oolongtheanin-3′-O-gallate of: 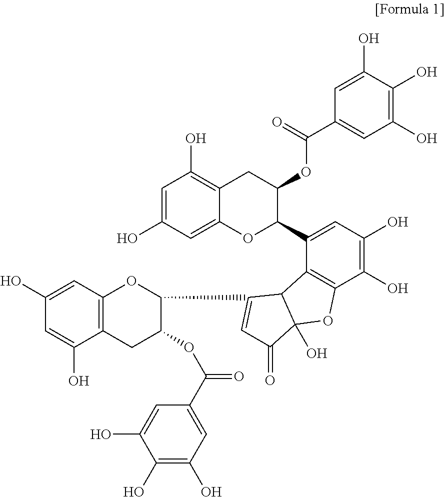
is added in an amount of 0.1 mg to 1000 mg per meal.
